(19) United States
(12) Patent Application Publication
(10) Pub. No.: US 2009/0163951 A1
(43) Pub. Date: Jun. 25, 2009

(54) MEDICAL DEVICES INCLUDING SUTURES WITH FILAMENTS COMPRISING NATURALLY DERIVED COLLAGENOUS MATERIAL

(76) Inventors: Sara Simmons, Sullivan, IN (US); Don Patterson, Bloomington, IN (US); Shyam SV Kuppurathanam, Bloomington, IN (US)

Correspondence Address: BRINKS HOFER GILSON & LIONE/CHICAGO/COOK, PO BOX 10395, CHICAGO, IL, 60610, US

(21) Appl. No.: 12/332,679

(22) Filed: Dec. 11, 2008

Related U.S. Application Data
(60) Provisional application No. 61/015,044, filed on Dec. 19, 2007.

(51) Int. Cl. A61B 17/04; A61F 2/04

(52) U.S. Cl. 606/229; 606/228; 623/23.7

(57) ABSTRACT

The present invention relates to implantable medical devices that include at least one suture attached to a device component, the suture including a naturally derived collagenous material. The present invention is also directed to sutures per se including filaments incorporating naturally derived collagenous material. The naturally derived collagenous material may be an extracellular matrix (ECM) material.

### RELATED APPLICATIONS

The present patent document claims the benefit of the filing date under 35 U.S.C. § 119(e) of Provisional U.S. Patent Application Ser. No. 61/015,044, filed Dec. 19, 2007, which is hereby incorporated by reference.

### BACKGROUND

1. Technical Field

The present disclosure relates to medical devices including sutures that include filaments that are made from, incorporate and/or are coated with naturally derived collagenous material, such as extracellular matrix (ECM) material, or compositions thereof. The present invention further relates to sutures per se that include filaments that are made from, incorporate and/or are coated with a naturally derived collagenous material, such as ECM material.

2. Background Information

Surgical sutures are well known medical devices in the art. The sutures may have braided (see, e.g., U.S. Pat. No. 5,019,093) or monofilament constructions, and may be provided in single-armed or double-armed configurations with a surgical needle mounted to one or both ends of the suture, or may be provided without surgical needles mounted. The sutures are used in a variety of conventional medical and surgical procedures to approximate tissue, affix or attach implants to tissue, assemble medical devices, etc.

Surgical sutures may be made from a variety of known polymeric bioabsorbable and nonabsorbable materials. For example, sutures are known to be made from aromatic polyesters such as polyethylene terephthalate, nylons such as nylon 6 and nylon 66, polyolefins such as polypropylene, silk, and other nonabsorbable polymers. In addition, sutures may be made from polymers and copolymers of p-dioxanone (also known as 1,4-dioxane-2-one), ε-caprolactone, glycolide, L(−)-lactide, D(+)-lactide, meso-lactide, trimethylene carbonate, and combinations thereof. Of particular utility are often polydioxanone homopolymer sutures.

Surgical sutures are typically available in a range of conventional sizes for a variety of conventional surgical procedures. The size of the suture for use in any particular application is dictated in part by the type of medical device elements or tissue to be sutured, the relative size of the medical device elements or tissue structure, as well as the forces that will be applied to the sutures by the approximated medical device elements or tissue after the surgical procedure has been completed. Similarly, the type of suture selected can be dictated by the procedure. For example, nonabsorbable sutures are typically used for applications such as cardiovascular, vascular, orthopedic, gastrointestinal and the like wherein a nonabsorbable suture is desired or required because a permanent or an extended period of fixation is required during the healing period, e.g., implantation of a heart valve prostheses. Bioabsorbable sutures are typically used for applications such as plastic surgery, skin fixation and certain soft tissue approximation, and the like. A bioabsorbable suture may be used when extended tissue approximation or fixation is not required as long as the suture maintains adequate strength during the healing period, and it is desirable to replace the suture with autologous tissue such as skin or soft tissue during the healing process.

Braided polyester sutures are useful in applications where a strong, nonabsorbable suture is needed to permanently repair tissue. These types of sutures are frequently used in cardiovascular surgery, as well as in ophthalmic and neurological procedures. Examples of commercially available braided polyester sutures are ETHIBOND EXCEL®, manufactured by Ethicon, Inc., TICRON® manufactured by Sherwood-Davis & Geck and TEVDEK® and POLYDEK® manufactured by Teleflex Medical, Limerick, Pa.

Examples of sutures prepared from biocompatible bioabsorbable polymers are well known in the art and are described, e.g., in U.S. Pat. Nos. 2,668,162; 2,703,316; 2,758,987; 3,225,766; 3,297,033; 3,422,181; 3,531,561; 3,565,077; 3,565,869; 3,620,218; 3,626,948; 3,636,956; 3,736,646; 3,772,420; 3,773,919; 3,792,010; 3,797,499; 3,839,297; 3,867,190; 3,878,284; 3,982,543; 4,047,533; 4,060,089; 4,137,921; 4,157,437; 4,234,775; 4,237,920; 4,300,565; 4,523,591, U.K. Patent No. 779,291; Gilding et al., Biocompatibility of Clinical Implant Materials, Vol. II, ch. 9: “Biodegradable Polymers” (1981). Synthetic biocompatible bioabsorbable multifilament sutures such as DEXON®, VICRYL®, and POLYSORB® are commercially available from Ethicon, Inc. (Somerville, N.J.) and United States Surgical (Norwalk, Conn.) are well known to those in the industry.

Other commercially available sutures fabricated from biocompatible non-bioabsorbable polymers, e.g., a polyester suture (SURGIDAC®, United States Surgical, Norwalk, Conn.) and a polyester braided suture (TICRON®, David & Geck, Danbury, Conn.) are also well known to those in the industry.

Various suture coating compositions are also well known in the art. For example, U.S. Pat. No. 4,027,676 discloses absorbable coating compositions for sutures. Other suture coatings are described, e.g., in U.S. Pat. Nos. 4,624,256; 4,190,720; 4,582,052; 4,605,730; 4,700,704; 4,705,820; 4,788,979; 4,791,929; 4,994,074; 5,047,048; 5,100,433; 5,352,515; 5,032,638; 4,711,241; and 4,201,216.

### SUMMARY

The present invention relates to implantable medical devices including sutures that include filaments that are made from, incorporate and/or are coated with naturally derived collagenous material, such as extracellular matrix (ECM) material, or compositions thereof. The present invention further relates to sutures per se that include filaments that are made from, incorporate and/or are coated with a naturally derived collagenous material, such as ECM material. Applicants discovered that the sutures that include filaments incorporating a naturally derived collagenous material surprisingly encourage attachment, or cell growth (i.e., tissue ingrowth or proliferation), in places of contact of the sutures with the tissue when used in a human body. The use of naturally derived collagenous materials in the sutures may provide the advantage, for example, by allowing for improved medical device fixation and sealing, or improved tissue healing.

In one embodiment, the invention relates to an implantable device comprising a device component; and at least one suture attached to the device component, the suture comprising a filament comprising a polymeric material and a naturally derived collagenous material.

In another embodiment, the invention relates to an implantable device comprising a device component; and at least one suture attached to the device component, the suture comprising a plurality of filaments, wherein the filaments comprise a polymeric material and a naturally derived collagenous material. The suture may be tied to the device component. The device may further be comprising a second device component, the suture attaching the first and the second device components to each other. The device component may comprise a stent. The device component may comprise a graft. The suture may be a running suture. The naturally derived collagenous material may be an extracellular matrix material selected from the group consisting of submucosa, renal capsule membrane, dermal collagen, dura mater, pericardium, fascia lata, serosa, peritoneum or basement membrane layers, liver basement membrane, intestinal submucosa, small intestinal submucosa, stomach submucosa, urinary bladder submucosa, and uterine submucosa. The filaments may be coated with the naturally derived collageous material. The naturally derived collagenous material may be made into at least one filament. The suture may be a braided suture. Some of the filaments may comprise the polymeric material and other filaments may comprise the naturally derived collagenous material. The filaments that comprise the polymeric material may form a core of the braided suture. The filaments that comprise the naturally derived collagenous material may be braided around the core of the braided suture. Some other filaments that comprise the polymeric material and the filaments that comprise the naturally derived collagenous material may be braided together around the core of the braided suture. Some of the filaments that comprise the naturally derived collagenous material may form a core of the braided suture and the filaments that comprise the polymeric material may be braided around the core of the braided suture. Some other filaments that comprise the naturally derived collagenous material and the filaments that comprise the polymeric material may be braided together around the core of the braided suture. Some filaments that comprise the polymeric material and some filaments that comprise the naturally derived collagenous material together may form a core of the braided suture. Some other filaments that comprise the polymeric material may be braided around the core of the braided suture. Some other filaments that comprise the naturally derived collagenous material may be braided around the core of the braided suture. Some filaments that comprise the polymeric material and some other filaments that comprise the naturally derived collagenous material may be braided together around the core of the braided suture. The suture may be coated to improve its surface lubricity and knot tiedown behavior. The suture may enhance adhesion of the device component to a tissue in place of contact of the suture with the tissue when used in a human body. The device may further include at least one therapeutic agent selected from the group consisting of antimicrobial agents, gentamycin sulphate, erythromycin, derivatized glycopeptides, growth factors, fibroblast growth factor, bone growth factor, epidermal growth factor, platelet-derived growth factor, macrophage-derived growth factor, alveolar-derived growth factor, monocyte-derived growth factor, magainin, carrier proteins, glycerol with tissue or kidney plasminogen activator; superoxide dismutase, tumor necrosis factor; colony stimulating factor, interferon, interleukin-2, and lymphokines.

In yet another embodiment, the invention relates to a suture comprising (i) a filament comprising a polymeric material and a naturally derived collagenous material; or (ii) a plurality of filaments, wherein the filaments comprise a polymeric material and naturally derived collagenous material. The naturally derived collagenous material may be an extracellular matrix material selected from the group consisting of submucosa, renal capsule membrane, dermal collagen, dura mater, pericardium, fascia lata, serosa, peritoneum or basement membrane layers, liver basement membrane, intestinal submucosa, small intestinal submucosa, stomach submucosa, urinary bladder submucosa, and uterine submucosa. The suture may be tied around at least one device component to promote tissue ingrowth or proliferation around the device upon placement of the device in a body. The suture may also be for suturing wounds, such as skin wounds. The suture may further include a needle.

### BRIEF DESCRIPTION OF THE DRAWINGS

FIGS. 1A-B are schematic illustrations of exemplary sutures of the present invention;

FIG. 2 is a schematic illustration of one embodiment of this invention;

FIG. 3 is a schematic illustration of another embodiment of this invention; and

FIG. 4 depicts an apparatus to deploy a prosthesis or prosthetic device of this invention.

FIG. 5 depicts a schematic illustration of still another embodiment of this invention.

### DETAILED DESCRIPTION OF THE DRAWINGS AND THE PRESENTLY PREFERRED EMBODIMENTS

The present disclosure relates to implantable medical devices including sutures that include filaments that are made from, incorporate and/or are coated with naturally derived collagenous material, such as extracellular matrix (ECM) material, or compositions thereof. The present invention further relates to sutures per se that include filaments that are made from, incorporate and/or are coated with a naturally derived collagenous material, such as ECM material.

Applicants discovered that the sutures that include filaments incorporating a naturally derived collagenous material surprisingly encourage attachment, or cell growth (i.e., tissue ingrowth or proliferation), in places of contact of the sutures with the tissue when used in a human body. The term “cell growth, proliferation and/or ingrowth in places of contact” refers to cellular proliferation and/or ingrowth onto or on all areas of the device where the suture comes in contact with the tissue so to surround the device and make the device hemocompatible. The term also refers to cellular proliferation and/or ingrowth onto or on all areas where the suture is used (e.g., so to surround a skin wound). Also, the sutures may promote ingrowth or proliferation of tissue to close punctures in the graft material created by the suturing process so as to prevent endoleaks. In addition, the sutures of this invention may accelerate or beneficially modify the healing process when the suture is applied to a wound or surgical site. Moreover, the attachment of the stent graft assembly to numerous points covering the exterior of the stent graft assembly could reduce stent graft migration in patients.

The sutures may be monofilament sutures or braided sutures. The monofilament sutures include at least one polymeric filament that incorporates (e.g., by coating or impregnation, or other suitable method) naturally derived collagenous material, such as extracellular matrix (ECM) material.

The braided suture may include a plurality of filaments, where the filaments comprise a polymeric material and a naturally derived collagenous material. The polymeric filaments may be coated with the naturally derived collagenous material.

The sutures may be attached (e.g., tied, wrapped around, etc.) to the components of the medical device, may be used for attaching components of the medical device to each other, or both.

In addition to their use with medical devices, the sutures described herein may have other numerous other applications in medicinal arts. For example, these sutures may also be used as surgical sutures in hernia repair, cardiovascular repair, cardiovascular valve implant attachments and other suitable applications (e.g., skin sutures or other external or topical sutures).

In the present context, the term “suture” means a thread or material. The thread or material may simply be attached to a part of a structure or may be used to secure two parts together. In one example, suture may mean any material that approximates and secures tissue of a living body. In another example, a suture may mean any material that secures components or elements of a medical device (i.e., device components) to each other, for example in an endoluminal prosthetic device, such as to secure two or more stents together or to secure stent(s) to a graft. In still another example, suture may also refer to the configuration of this material in a loop, for example, the material securing a portion of a stent to a graft. A suture may be made by looping material through the graft and around the stent or an apex of a stent. In certain embodiments, a suture may be made by looping material around a device component, such as a stent.

In the present context, a suture may be secured with a knot, and where there is a suture, there is at least one loop of thread or material securing a portion of a stent to a graft, and a knot securing the suture. A knot may be tied, intertwining the ends of the suture in such a way that they will not be easily separated. A suture thus has a knot and may have more than one knot. In some cases, or in many cases, an apex of a stent may be secured to the graft by two loops of suture or thread through the graft and also through or around the apex of the stent, and then secured with one knot or more than one knot, in essence tying the ends of the suture. Knots may be locking knots, preferred for apices, or overthreaded knots, preferred along the length of the strut or for other applications. Knots in the stent graft may include any other useful or desired knots and are not limited to these types.

If the ends of the knot are not cut, the thread or suture may be led by a needle or other mechanical device to the next point on the device where a suture is desired. The thread or material that joins the first suture to the next suture may be called a running suture, because the thread or suture “runs” between the sutures. Exemplary running sutures and medical device incorporating same were previously described in U.S. Pub. No. 2005/0159803 A1, which is incorporated herein by reference in its entirety.

A “stitch” is a single suture and includes at least one knot.

Sutures for stents are typically not single sutures or single stitches, but typically include two loops through the graft and around the stent or apex that is being joined. The sutures may then be affixed with one or more knots. Multiple stitches for attaching two parts together were previously described in U.S. Pub. No. 2005/0159804 A1, which is incorporated herein by reference in its entirety.

The term suture as used herein is intended to embrace:
a) sutures that include a single filament (i.e., monofilament sutures), where the filament includes polymeric material which may be of non-absorbable and/or bioabsorbable varieties and incorporates a naturally derived collagenous material, such as ECM; or
b) sutures that include a plurality of filaments, where the filaments comprise polymeric material, which may be of non-absorbable and/or bioabsorbable varieties, and a naturally derived collagenous material, such as ECM.

The sutures of this invention may be unbraided or braided sutures. The suture may include a needle mounted on either end of the suture.

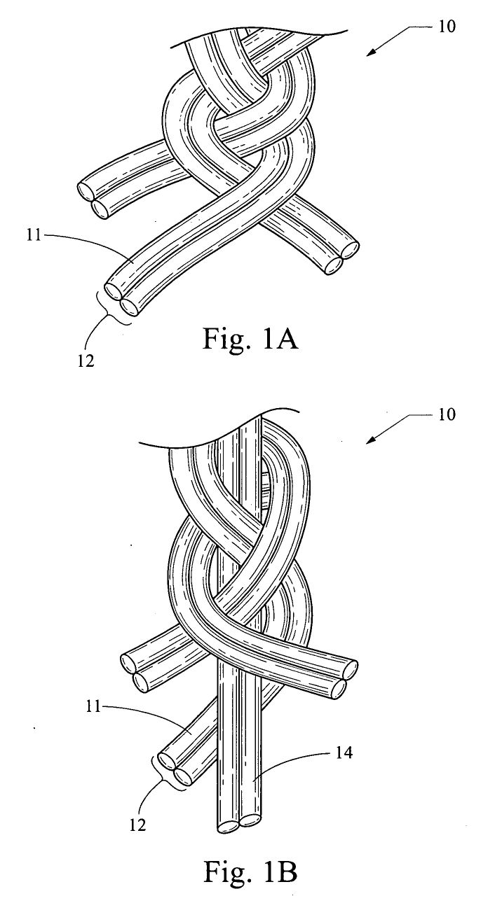

The suture for use with a medical device of this invention is preferably a braided suture 10 shown, for example in FIGS. 1A-B. The terms “braid” or “braided” as applied to the sutures described herein refer to an arrangement of discrete units, or bundles, denominated “sheath yarns” 12, made up of individual filaments 11 with individual sheath yarns interlocking or interlacing each other in a regular criss-cross pattern or other suitable pattern.

The term “filament” refers to a single, long, thin flexible structure of a non-absorbable or absorbable polymeric material, a naturally derived collagenous material, such as ECM, or both (e.g., polymeric filament coated with ECM). It may be continuous or staple. “Staple” is used to designate a group of shorter filaments which are usually twisted together to form a longer continuous thread. An absorbable filament is one which is absorbed, that is digested or dissolved, in living mammalian tissue.

A “thread” is a plurality of filaments, either continuous or staple, twisted together.

A “strand” is a plurality of filaments or threads twisted, plaited, braided, or laid parallel to form a unit for further construction into a fabric, or used per se, or a monofilament of such size as to be woven or used independently.

A filament or filaments of the suture, or any part thereof, may be made of a biocompatible polymeric material, forming polymeric filament(s), suitable for the application, including but not limited to, monofilament polyester or braided multi-filament polyester, nylon, polyaramid, polypropylene, and polyethylene. In some embodiments, the polyester employed to make polymeric filaments is polyethylene terephthalate (PTE). For example, Green, braided polyester 4-0 suture material would be preferred for sutures for attaching external stents to grafts, while monofilament suture material (5-0 Blue Polypropylene, for instance) would be preferred for sutures for attaching internal and anchoring stents to grafts. The polyester 4-0 suture material is nonabsorbable and has limits of 0.150 to 0.199 mm (metric size 1.5). Suture materials are commercially available from a number of companies, including Genzyme Biosurgery, Fall River, Mass.; Teleflef Medical, Limerick, Pa.; Ethicon Inc, Cornelia, Ga., among others.

In certain embodiments, these polymeric filaments or any part thereof, may be impregnated or coated with, or otherwise incorporate a reconstituted or naturally derived collagenous material, such as ECM material. The term “incorporate” will be used for simplicity to encompass coating, applying, waving in, impregnating, making, forming or otherwise incorporating the ECM into filaments of a suture.

Other filaments of the suture for use in this invention or any part thereof, are made from, are impregnated or coated with, or otherwise incorporate a reconstituted or naturally derived collagenous material, such as ECM material.

Applicants discovered that it may be advantageous to incorporate into a suture or part thereof a remodelable, and particularly a remodelable collagenous material. Such remodelable collagenous material can be provided, for example, by collagenous materials isolated from suitable tissue source from a warm-blooded vertebrate, and especially a mammal. Such isolated collagenous materials may be processed so as to have remodelable properties and promote cellular invasion and tissue infiltration. Remodelable materials may be used in this context to promote cellular growth or ingrowth at places of contact with tissue, while optionally containing others materials.

Reconstituted or naturally-derived collagenous materials are incorporated into the sutures of the present invention. Such materials that are at least bioresorbable will provide advantage in the present invention, with materials that are bioremodelable and promote cellular invasion and ingrowth providing particular advantage. For example, such materials may provide for improved device fixation and sealing.

Suitable bioremodelable materials may be provided by collagenous extracellular matrix materials (ECMs) possessing biotropic properties, including in certain forms angiogenic collagenous ECMs. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa.

As prepared, the submucosa material and any other ECM used may optionally retain growth factors or other bioactive components native to the source tissue. For example, the submucosa or other ECM may include one or more growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a bioactive component that induces, directly or indirectly, a cellular response such as a change in cell morphology, proliferation, growth, protein, or gene expression.

Submucosa or other ECM materials may be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with specific staining. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the infiltration of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (7):833-839 (2001). When combined with a fluorescence microangiography technique, this model can provide both quantitative and qualitative measures of angiogenesis into biomaterials. C. Johnson et al., Circulation Research 94(2):262-268 (2004).

Further, in addition or as an alternative to the inclusion of native bioactive components, non-native bioactive components such as those synthetically produced by recombinant technology or other methods, may be incorporated into the submucosa or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics or thrombus-promoting substances such as blood clotting factors, e.g., thrombin, fibrinogen, and the like. These substances may be applied to the ECM material as a premanufactured step, immediately prior to the procedure (e.g., by soaking the material in a solution containing a suitable antibiotic such as cefazolin), or during or after delivery of the material in the patient.

Submucosa or other ECM tissue used in the invention is preferably highly purified, for example, as described in U.S. Pat. No. 6,206,931, which is incorporated by reference herein. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosa tissue used in the present invention.

Preferred type of submucosa for use in this invention is derived from the intestines, more preferably the small intestine, of a warm blooded vertebrate; i.e., small intestine submucosa (SIS). SIS is commercially available from Cook Biotech, West Lafayette, Ind.

Preferred intestine submucosal tissue typically includes the tunica submucosa delaminated from both the tunica muscularis and at least the luminal portions of the tunica mucosa. In one example the submucosal tissue includes the tunica submucosa and basilar portions of the tunica mucosa including the lamina muscularis mucosa and the stratum compactum. The preparation of intestinal submucosa was described in U.S. Pat. No. 4,902,508, and the preparation of tela submucosa was described in U.S. Pat. No. 6,206,931, both of which are incorporated herein by reference. The preparation of submucosa was also described in U.S. Pat. No. 5,733,337 and in 17 Nature Biotechnology 1083 (November 1999); and WIPO Pub. WO 98/22158, which is the published application of PCT/US97/14855. Also, a method for obtaining a highly pure, delaminated submucosa collagen matrix in a substantially sterile state was previously described in U.S. Pub. No. 2004 0180042 A1, disclosure of which is incorporated by reference.

The ECM material for use in the present invention may be processed to provide preferred shape or form of the ECM material. For example, the ECM material may take many shapes and forms, such as string or fiber-like, filament, thread, coiled; helical; spring-like; randomized; branched; sheet-like; tubular; spherical; fragmented; powdered; ground; sheared; fluidized; sponge-like; foam-like; and solid material shape. Filament or thread-like forms of the ECM materials are preferred for use in this invention. The filaments made from ECM material may be used to form the sutures used with the device of this invention. In some embodiments, a fluidized form of the ECM material may be preferred, for example for use as a coating for polymeric or ECM filaments. A fluidized form of the ECM material may also be used to impregnate the polymeric filaments.

While naturally derived biomaterials, particularly bioremodelable materials like SIS described above, are generally preferred for use in this invention, synthetic materials, including those into which growth factors are added to make them bioremodelable, are also within the scope of this invention.

As illustrated in FIGS. 1A-B, the braided sutures 10 for use in this invention may have two configurations. In some embodiments, the filaments 11 form sheath yarns 12, which are braided together into a “braid” (FIG. 1A). In other embodiments, as illustrated in FIG. 1B, the filaments 11 form sheath yarns that may be braided into sutures using conventional braid construction having sheath yarns 12 and a core 14 (FIG. 1B). Typical braid constructions are disclosed, for example, in U.S. Pat. Nos. 5,019,093 and 5,456,697, which are incorporated herein by reference in their entirety.

Both types of braided sutures include a plurality of filaments, where some of the filaments comprise a polymeric material and some other filaments comprise a naturally derived collagenous material, such as ECM material.

In certain embodiments, the ECM material is made into yarn filaments that form a core 14 of the braided suture. In another embodiment, the ECM material is made into filaments that form the sheath yearns 12 of the braided suture. In yet another embodiment, the ECM material is made into yarn filaments that may form both the core as well sheath yarns of the braided suture provided that some other filaments of the braided suture are polymeric filaments. In yet further embodiment, ECM material may be used to coat polymeric filaments for use in the braided sutures of the invention. In certain embodiments, the ECM material is made into yarn filaments that form a core 14 and/or sheath yarns 12 of the braided suture 10 for use in this invention.

Exemplary configurations of the braided sutures having sheath yarns 12 and a core 14 for use with the device of the present invention are shown in Table I below. Other configurations are also contemplated.

TABLE I

| Suture | Core | Sheath yarns |
|---|---|---|
| Example 1: | ECM filaments | Polymeric filaments |
| Example 2: | ECM filaments | Polymeric filaments coated |
| | | with ECM |
| Example 3: | ECM filaments | ECM filaments and polymeric |
| | | filaments |
| Example 4: | ECM filaments | ECM filaments and polymeric |
| | | filaments coated with ECM |
| Example 5: | ECM filaments and | ECM filaments |
| | polymeric filaments | |
| Example 6: | ECM filaments and | Polymeric filaments |
| | polymeric filaments | |
| Example 7: | ECM filaments and | Polymeric filaments coated |
| | polymeric filaments | with ECM |
| Example 8: | ECM filaments and | ECM filaments and polymeric |
| | polymeric filaments | filaments |
| Example 9: | ECM filaments and | ECM filaments and polymeric |
| | polymeric filaments | filaments coated with ECM |
| Example 10: | Polymeric filaments | ECM filaments |
| Example 11: | Polymeric filaments | ECM filaments and polymeric |
| | | filaments |
| Example 12: | Polymeric filaments | ECM filaments and polymeric |
| | | filaments coated with ECM |
| Example 13: | Polymeric filaments coated | ECM filaments |
| | with ECM | |
| Example 14: | Polymeric filaments coated | Polymeric filaments |
| | with ECM | |
| Example 15: | Polymeric filaments coated | ECM filaments and polymeric |
| | with ECM | filaments |
| Example 16: | Polymeric filaments coated | ECM filaments and polymeric |
| | with ECM | filaments coated with ECM |
| Example 17: | Polymeric filaments coated | Polymeric filaments coated |
| | with ECM | with ECM |

Referring to FIG. 1B, for instance, when reading Example 1 above, the core 14 of the braided suture comprises yarn filaments made from the ECM material and polyester filaments 11 are braided around the core 14 made from the ECM filaments.

As noted in Table I above, the sheath yarns, the core, or both can comprise a combination of polymeric as well as ECM filaments. Some, portions of, or entire filaments (polymeric or ECM) may be coated, for example with ECM material.

Preferred braid constructions apart from the material of its constructions, also include: (1) overall suture denier; (2) the pattern of the interlocking yarns expressed in pick count, which is to say, the number of crossovers of individual sheath yarns per linear inch of suture; (3) the number of sheath yarns comprising the braid; (4) the denier of the individual filaments comprising each sheath yarn; and, (5) the denier of the core. Some of these, such as the denier of the individual filaments, also relates to monofilament sutures.

(1) Overall Denier of the Suture

The overall denier of the preferred braided suture for use in this invention can vary from about 50 to about 4000. Within this range, the ranges of overall denier for particular sutures are: from above about 125 to about 200 denier; from above about 200 to about 300 denier; from above about 300 to about 500 denier; from above about 500 to about 800 denier; from above about 800 to about 1200 denier; from above about 1200 to about 2000 denier; and, from above about 2000 to about 4000 denier. Other alternative denier ranges are also contemplated.

(2) Pattern of the Interlocking Sheath Yarns (Pick Count)

The term “pick count” as applied to a braided suture construction refers to the number of crossovers of sheath yarns per linear inch of suture and, together with the overall denier of the suture, the denier of the individual filaments constituting a sheath yarn and the number of sheath yarns employed, defines the principal construction characteristics of a braided suture.

For a preferred suture of any range of overall denier, pick count can vary from about 50 to about 100 crossovers/inch with about 55-80 crossovers/inch being preferred. For preferred sutures constructed within any range of overall denier, as larger numbers of sheath yarns are employed, the pick-count for acceptable sutures will also increase within the above ranges. For a preferred suture of a particular range of denier and number of sheath yarns, pick count is advantageously established to achieve a balance in the properties desired. For preferred sutures of any specific denier range and number of sheath yarns, it is preferable to have as low a pick count as possible in order to achieve optimum surface smoothness.

(3) The Number of Sheath Yarns

In the preferred suture, the number of sheath yarns bears some relation to overall suture denier, the number generally increasing with the weight of the suture. Thus, across the range of suture weight (denier) indicated above, the preferred braided suture can be constructed with from about 4 up to as many as about 36 individual sheath yarns constructed from individual filaments having the deniers for example, in the range from about 0.2 to about 6, in the range of from about 1.2 to about 3.4, or in the range of from about 1.4 to about 3.1.

Table II below sets forth broad and preferred ranges for the numbers of sheath yarns which are suitable for the construction of preferred braided sutures of various ranges of overall denier. The pick counts of the preferred sutures vary from about 50 to about 100 and deniers of individual filaments vary from about 0.2 to about 6.0 for the broad range of number of sheath yarns and the pick counts vary from about 55 to about 80 and the deniers of individual filaments vary from about 0.8 to about 3.0, and advantageously from about 1.0 to about 1.8, for the preferred range of number of sheath yarns.

TABLE II
Number of Sheath Yarn Related to Suture Denier

| | | | Number of |
|---|---|---|---|
| | | Number of | Sheath Yarns |
| Overall Suture | | Sheath Yarns | (Preferred |
| Denier | Suture Size | (Broad Range) | Range) |
| Greater than | 6/0 | 4-16 | 6-14 |
| about 125 to | | | |
| about 200 | | | |
| Greater than | 5/0 | 4-16 | 6-14 |
| about 200 to | | | |
| about 300 | | | |
| Greater than | 4/0 | 10-20 | 12-14 |
| about 300 to | | | |
| about 500 | | | |
| Greater than | 3/0 | 14-20 | 14-18 |
| about 500 to | | | |
| about 800 | | | |
| Greater than | 2/0 | 16-32 | 20-30 |
| about 800 to | | | |
| about 1200 | | | |
| Greater than | 0 | 20-36 | 24-32 |
| about 1200 to | | | |
| about 2000 | | | |
| Greater than | 1, 2 | 20-36 | 24-32 |
| about 2000 to | | | |
| about 4000 | | | |

While the sheath yarns need not be twisted, it is generally preferred that they be provided with a twist so as to minimize snagging during braid construction. Alternatively, the sheath yarns can be air entangled.

(4) Individual Filament Denier

In one embodiment, the individual filaments comprising each sheath yarn of the braided (or monofilament) suture can vary in weight. For smaller sutures, i.e., sutures having an overall suture denier of less than about 300, the individual sheath filaments can vary in weight from about 0.2 to about 3.0 denier, and preferably from about 1.0 to about 1.8 denier. For larger sutures, i.e., sutures having an overall suture denier of greater than about 300, individual sheath filaments can vary in weight from about 0.2 to about 6.0 denier, preferably from about 0.8 to about 3.0 denier, and more preferably from about 1.0 to about 1.8 denier. The number of such filaments present in a particular sheath yarn will depend on the overall denier of the suture as well as the number of sheath yarns utilized in the construction of the suture. Table III sets forth some typical numbers of filaments per sheath yarn for both the broad and preferred ranges of filament weight:

TABLE III
Number of Filaments per Sheath Yarn

| Approximate | Approximate | |
|---|---|---|
| Minimum | Maximum | Filament Denier |
| 45 | 450 | 0.2 |
| 15 | 150 | 0.5 |
| 5 | 50 | 1.5 |
| 3 | 40 | 1.8 |
| 1 | 15 | 6.0 |

As discussed previously, the individual filaments of the braided suture may be fabricated from a polymeric material, such as bioabsorbable polymer derived at least in part from one or more monomers selected from the group consisting of glycolic acid, glycolide, lactic acid, and lactide. In some embodiments, the individual filaments may be fabricated from a polymeric material, such as a non-absorbable material, e.g., cotton, silk, polyamide or polyolefin. In other embodiments, the individual filaments of the braided suture may incorporate, are made from, or are coated with naturally derived collagenous material, such as the ECM material.

(5) Core

The core of the braided suture may be manufactured in a separate operation and may be assembled from a plurality of individual yarns, e.g., from about 2 to about 1500, and preferably from 3 to about 1323 yarns. Preferably, the core is fabricated from the ECM filaments and/or polymeric filaments that incorporate ECM (for example, by coating or impregnation).

In one embodiment, the core may be cabled. For example, each yarn comprising the core may given a twist in one direction, the “front” direction, the twisted yarns then being combined into a core which is then twisted in the opposite direction, the “back” direction, to provide the cabled core unit around which the remainder of the suture is constructed. Depending upon the material used to construct the core, it may be desirable to heat set and/or stretch the core in a known manner prior to final assembly of a braided suture incorporating the cabled core. Examples of cabled core were previously described in U.S. Pat. No. 5,456,697, which is incorporated herein by reference.

The denier of the individual yarns comprising the core is not particularly critical and can range in most cases from about 10 to about 100 and preferably from about 20 to about 70.

The overall denier of the core may be determined by the number and individual deniers of the core yarns from which the core is constructed. For many suture constructions, core denier will range from about 20 to about 80 and preferably from about 25 to about 50 in the smallest size suture and from about 800 to about 2400 and preferably from about 1000 to about 2200 in the largest size suture. In order to increase the total core denier, it is contemplated that for larger suture cores it may be desirable to ply two or more yarns together, preferably before front twisting of the yarns.

Table IV below provides examples of some typical core deniers for sutures of various deniers.

TABLE IV
Core Denier Related to Suture Denier

| | | | Denier of Core |
|---|---|---|---|
| Overall Suture | | Denier of Core | (Preferred |
| Denier | Suture Size | (Broad Range) | Range) |
| Greater than | 6-0 | 20-80 | 25-50 |
| about 125 to | | | |
| about 200 | | | |
| Greater than | 5-0 | 30-100 | 50-80 |
| about 200 to | | | |
| about 300 | | | |
| Greater than | 4-0 | 80-150 | 80-120 |
| about 300 to | | | |
| about 500 | | | |
| Greater than | 3-0 | 150-300 | 180-280 |
| about 500 to | | | |
| about 800 | | | |
| Greater than | 2-0 | 250-700 | 350-650 |
| about 800 to | | | |
| about 1200 | | | |
| Greater than | 0 | 400-1200 | 500-1000 |
| about 1200 to | | | |
| about 2000 | | | |
| Greater than | 1, 2 | 800-2400 | 1000-2200 |
| about 2000 to | | | |
| about 4000 | | | |

The entire suture (either braided or unbraided) or individual filaments of the suture of the present invention may further be coated to improve surface lubricity, knot tiedown behavior, and so forth. A variety of suture coating compositions proposed for either or both purposes is known in the art, e.g., those disclosed in U.S. Pat. Nos. 4,047,533; 4,027,676; and 4,043,344.

In addition, the suture of this invention may be provided with a therapeutic agent, which will be deposited at the sutured site. The therapeutic agent can be chosen for its antimicrobial properties, capability for promoting wound repair and/or tissue growth or for specific indications such as thrombosis.

Antimicrobial agents such as broad spectrum antibiotics (gentamycin sulphate, erythromycin or derivatized glycopeptides), which are slowly released into the tissue can be applied in this manner to aid in combating clinical and sub-clinical infections in a surgical or trauma wound site. To promote wound repair and/or tissue growth, one or several growth promoting factors can be introduced into the suture, e.g., human growth factors such as fibroblast growth factor, bone growth factor, epidermal growth factor, platelet-derived growth factor, macrophage-derived growth factor, alveolar-derived growth factor, monocyte-derived growth factor, magainin, carrier proteins, and so forth. Some therapeutic indications are: glycerol with tissue or kidney plasminogen activator to cause thrombosis, superoxide dismutase to scavenge tissue damaging free radicals, tumor necrosis factor for cancer therapy or colony stimulating factor and interferon, interleukin-2 or other lymphokine to enhance the immune system.

The suture material may be attached to a hollow needle used to thread the suture through the tissue or elements of the medical device, such as a graft, thus attaching two tissues together or two elements of the medical device, such as a stent to a graft, respectively. Once the needle is attached, the suture may then be packaged and sterilized with, for example, ionizing radiation, ethylene oxide, or the like.

### Medical Devices

The sutures of this invention may be used with various medical devices. In certain embodiments, the suture may be attached to a device component. In certain other embodiments, the suture attaches two or more device components together.

The term “device component” refers to any element, or part thereof, of a medical device, such as a stent, graft, stent graft, barb, balloon, catheter and any other suitable element, or part thereof that may be placed in a body lumen.

The term “stent” refers to a plurality of struts and joints or apices between the struts.

A “graft” refers to a flexible material that can be attached to a support frame, for example to form a stent graft. A graft material can have any suitable shape, but it preferably forms a tubular prosthetic vessel. A graft material can be formed from polyester or any other suitable biocompatible material. These materials may include, but are not limited to, polyester, polyurethane, polyethylene, polypropylene, and polytetrafluoroethylene, as well as other fluorinated polymer products. A preferred material is polyester, woven in a twill pattern available from Vascutek Ltd. Of Scotland. Biomaterials may also be used, such as collagen or reconstituted or naturally derived collagenous materials, including ECM materials, which were described above. One example is porcine SIS material, which may be remodeled into repair tissue for the human body. SIS is commercially available from Cook Biotech, Bloomington, Ind.

In one embodiment, the invention is an implantable device that includes at least one device component and at least one suture attached to the device component. The suture includes a single filament comprising a polymeric material and a naturally derived collagenous material.

In another embodiment, the invention is an implantable device that includes at least one device component and at least one suture, the suture comprising a plurality of filaments, wherein the filaments comprise a polymeric material and a naturally derived collagenous material. Preferably, the suture is a braided suture.

The suture may be attached, for example by tying, to a single device component. For example, the suture may be tied around a stent or tied or wrapped around a barb element(s) of a stent, such as an anchoring stent.

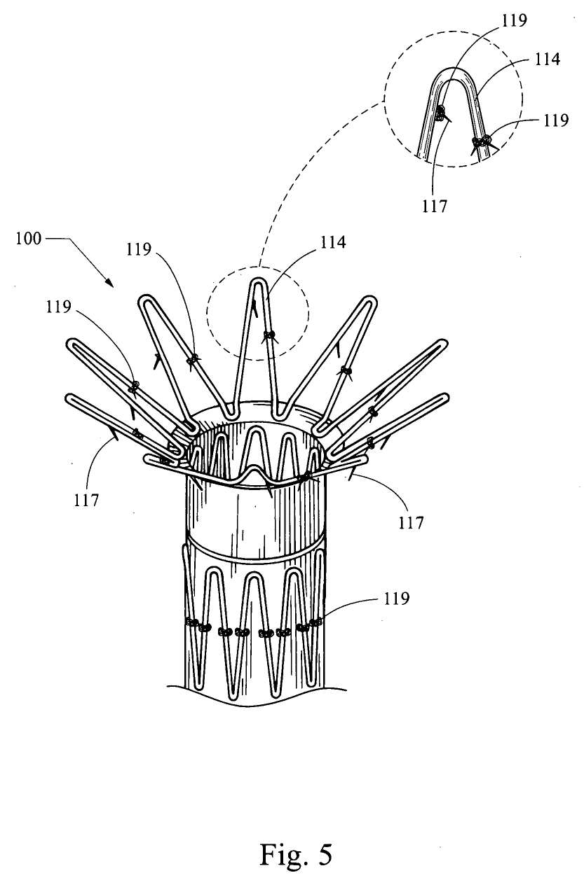

As illustrated in FIG. 5, sutures 119 that include filaments that incorporate naturally derived collagenous material, such as ECM, may be tied or wrapped around any or multiple part(s) of the various components or elements of a medical device 100. In one example, the suture 119 may be tied or wrapped on either side of the barb element(s) 117 present on the anchoring stent 114. This is to promote or encourage attachment, or cell growth (i.e., tissue ingrowth or proliferation), in places of contact of the sutures with the tissue when used in a human body, i.e., at the anchoring location.

In certain other embodiments, the sutures that include filaments that incorporate naturally derived collagenous material, such as ECM may be used for example, for attaching medical device components to each other to form a medical device assembly, such as a stent graft assembly, for various medical applications, including for example, aortic aneurysm and dissection treatment, hernia repair, cardiovascular repair, cardiovascular implant attachments and for other suitable applications.

Specifically, in certain embodiments, the invention is an implantable medical device comprising a first and a second device components and at least one suture attaching the first and the second device components to each other, the suture comprising a filament comprising a polymeric material and a naturally derived collagenous material; or a plurality of filaments, wherein the filaments comprise a polymeric material and a naturally derived collagenous material.

An implantable medical device may be formed by, for example, attaching a stent to a graft with the sutures described herein. At least one apex of the stent may be attached to the graft.

In making the sutures at the apices described below, suturing may begin at any convenient point.

In one embodiment, at least two sutures may share a penetration in the graft, which more firmly anchors the stent to the graft, preventing separation of the stent from the graft. By using at least one opening or penetration in the graft for more than one suture, the number of penetrations or openings in the graft may be minimized. Multiple stitches for attaching stent to graft were previously described in U.S. Pub. No. 2005/0159804 A1, which is incorporated herein by reference in its entirety.

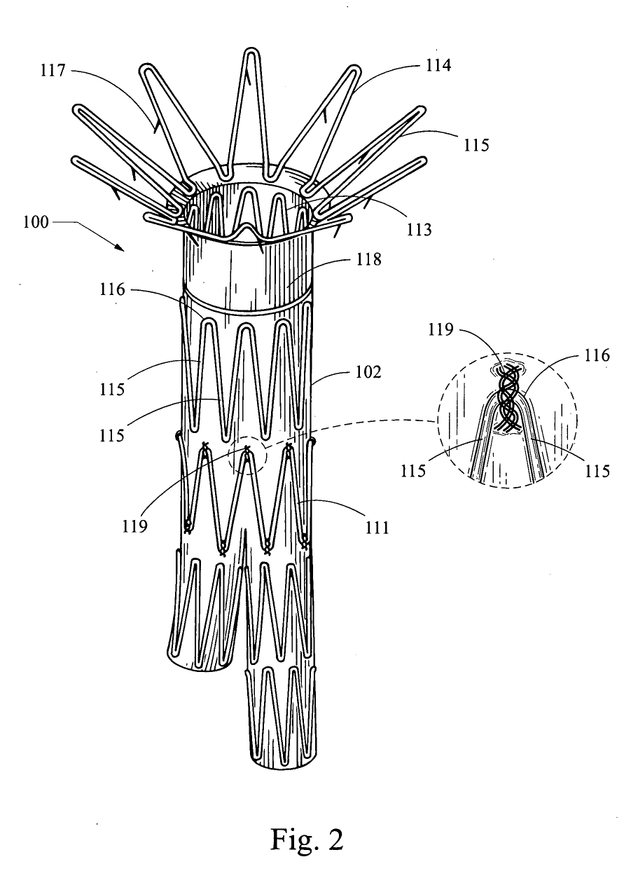

In one embodiment, the invention is a medical device presented in FIG. 2. The device 100 comprises a graft 102 made for example, from polyester or other graft material suitable for use in the human body, which were mentioned above. In one embodiment, the device 100 also includes at least one external stent 111. The device may also include at least one anchoring stent 114 and an internal stent 113. These stents may be in the form of zigzag stents, that is, a continuous chain of struts 115 and joints between the struts, or apices 116. Zigzag stents, sold under the trademark Cook-Z® and stent grafts for abdominal aortic aneurysm (AAA) repair, made by Cook Incorporated, Bloomington, Ind., are an example of commercial embodiments. Serpentine stents, having curved struts, may also be used.

In addition, anchoring stent 114 may include anchors 117, small barbs or hooks that will anchor the stent to the internal wall of a body lumen, such as a wall of an aorta. Anchoring stents are oriented so that they may easily attach to the body lumen when they are placed inside a patient. The barbs are preferably attached to struts 115 by welding, soldering, or other permanent attachment. The barb is preferably attached by several coils at a first pitch or spacing, and a final coil at a greater pitch than the first coils, to increase the fatigue life of the barbs.

The stents may be made from one or more of several materials. The preferred materials include, but are not limited to, stainless steal, titanium, and shape memory materials, such as Nitinol. These stents are desirably in a form of a zigzag stent, a continuous chain of struts and intersections where the struts join. As noted above, anchoring stent 114 may have one or more anchors 117, or small barbs, for securing the anchoring stent in a body lumen or passageway. Radially compressible, self-expanding stents are preferred. PCT Publication WO 98/53761, hereby incorporated by reference in its entirety, discloses a number of details concerning stents, stent grafts, and a method of delivering and/or implanting stent grafts into the human body.

The graft, as discussed above, should be suitable for placement inside a person, although these grafts are not limited to uses for humans and may also be used for animals. In this embodiment, the graft 102, as depicted in FIG. 2, may also include a short cuff 118. This cuff doubles the thickness of the graft and thus reinforces the graft in what may be the area of greatest stress. The end of the cuff forms a continuous band of fuzzy, frayed material (not shown). The fraying encourages ingrowth of tissue to the graft. A cuff, for example, of from about 0.050 inches to about 0.50 inches long may be used.

In one embodiment, an external stent 111 may be secured to graft using sutures 119, preferably braided sutures, through penetrations in the graft. In one embodiment, the external stent 111 may be secured to the graft 102 with more than one braided suture or stitch 119 in order to strengthen the its attachment to the graft. As mentioned previously, in order to minimize the number of penetrations in the graft, it may be desirable for the sutures to share penetrations or openings in the graft to the greatest extent possible. Using multiple stitches or sutures for attaching stent to graft was previously described in U.S. Pub. No. 2005/0159804 A1, which is incorporated herein by reference.

Similarly, anchoring stent(s) 114 may be secured to graft 102 using sutures 119 through penetrations in the graft (not shown). In one embodiment, the anchoring stent 114 may be secured to graft 102 with more than one stitch or suture 119 in order to strengthen its attachment to the graft. In order to minimize the number of penetrations in the graft, it may be desirable for the braided sutures to share penetrations or openings in the graft to the greatest extent possible. Note that the sutures are shown greatly exaggerated for clarity in all of the drawings.

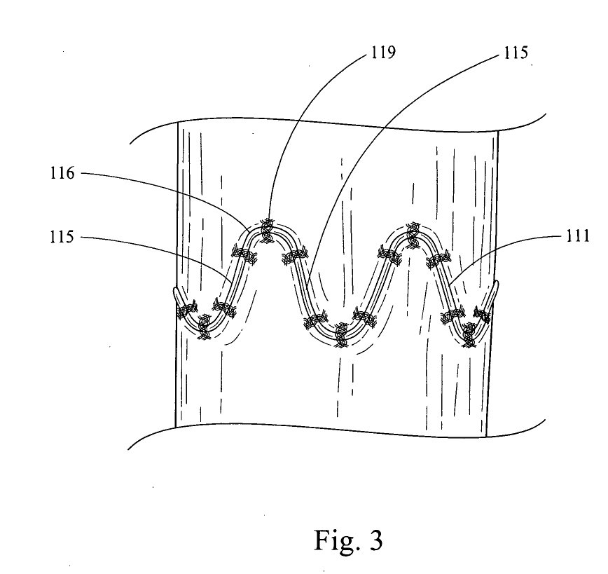

In one embodiment illustrated in FIG. 3, each segment of the external stent 111 includes two struts 115 and apex 116. In this embodiment, it is desired to secure the external stent 111 with at least two or more than one stitch per apex or segment.

There are many ways of practicing the invention. For instance, each bottom apex in the anchoring stent may be attached to the graft with multiple sutures described herein however, it is not necessary to so reinforce all the apices. If it is desired to add stitches or sutures in only one apex or a portion of the periphery of the stent or graft, that may also be done. In addition, the openings or penetrations may be prepared in advance in the graft, such as by punching or other preparatory method for forming openings or penetrations.

Each suture or stitch preferably includes at least one knot, so that once the suture is made, the suture will be secure in its position and will not move.

The sutures used for securing or attaching the device elements to each other include or otherwise incorporate a reconstituted or naturally derived collagenous material, such as ECM material described above. The presence of the ECM material in the suture surprisingly may encourage attachment, or cell growth, of stent graft to the inner wall of a body lumen, such as aorta, at all points where the braided suture comes in contact with the inner wall. The attachment of the stent graft to numerous points of the body lumen may prevent stent graft migration in patients and enhance anchoring of the stent to the internal wall of a body lumen. The presence of the ECM material in the suture may also increase the strength of the “seam” that is used as a permanent implant in humans.

The device of this invention may be deployed into a body lumen according to methods known in the art.

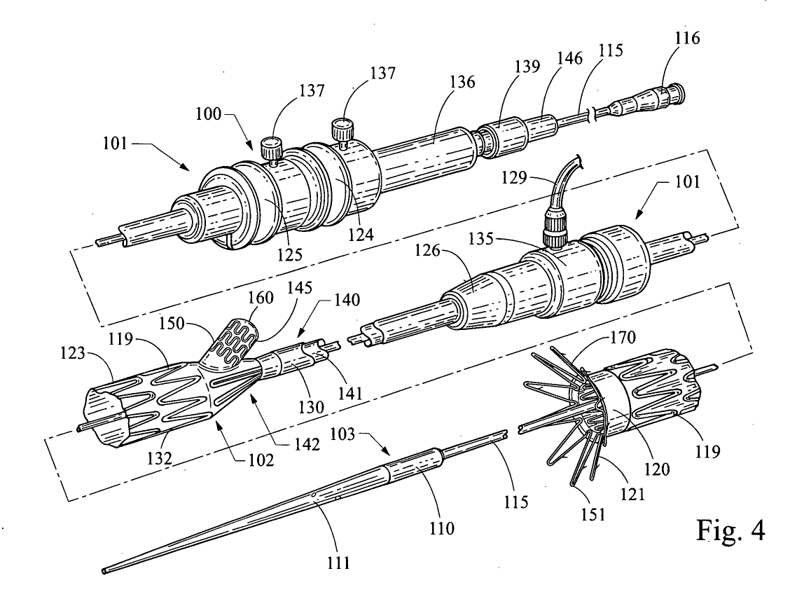

In one example, FIG. 4 shows a self-expanding bifurcated prosthesis 120, a self-expanding tubular prosthesis 150, and an endovascular deployment system 100, also known as an introducer 100, for deploying the prosthesis 120 in a lumen of a patient during a medical procedure. These items are each described in greater detail in PCT application WO 98/53761, which is incorporated herein by reference in its entirety. The terms “proximal” and “proximally” are used for a position or direction towards the patient's heart and the terms “distal” and “distally” are used for a position or direction away from the patient's heart.

The bifurcated prosthesis 120 has a generally inverted Y-shaped configuration. The prosthesis 120 includes a body 123, a shorter leg 160 and a longer leg 132. The bifurcated prosthesis 120 comprises a tubular graft material, such as polyester, with self-expanding stents 119 attached thereto. The self-expanding stents 119 cause the prosthesis 120 to expand following its release from the introducer 100. The prosthesis 120 also includes a self-expanding zigzag stent 121 that extends from its proximal end. The self-expanding zigzag stent 121 has distally extending barbs 151. When it is released from the introducer 100, the self-expanding zigzag stent 121 anchors the barbs 151, and thus the proximal end of the prosthesis 120, to the lumen of the patient.

The self-expanding tubular prosthesis 150 is similar to the bifurcated prosthesis 120, but has a unitary (i.e., non-bifurcated) lumen. The tubular prosthesis 150 also comprises a tubular graft material, such as polyester, having self-expanding stents attached thereto. The tubular prosthesis 150 is configured to connect to the shorter leg 160 of the bifurcated prosthesis 120.

The introducer 100 includes an external manipulation section 101, a distal attachment region 102 and a proximal attachment region 103. The distal attachment region 102 and the proximal attachment region 103 secure the distal and proximal ends of the prosthesis 120, respectively. During the medical procedure to deploy the prosthesis 120, the distal and proximal attachment regions 102 and 103 will travel through the lumen to a desired deployment site. The external manipulation section 101, which is acted upon by a user to manipulate the introducer, remains outside of the patient throughout the procedure.

The proximal attachment region 103 of the introducer 100 includes a cylindrical sleeve 110. The cylindrical sleeve 110 has a long tapered flexible extension 111 extending from its proximal end. The flexible extension 111 has an internal longitudinal aperture (not shown). This longitudinal aperture facilitates advancement of the tapered flexible extension 111 along an insertion wire (not shown). The longitudinal aperture also provides a channel for the introduction of medical reagents. For example, it may be desirable to supply a contrast agent to allow angiography to be performed during placement and deployment phases of the medical procedure.

A thin walled metal tube 115 is fastened to the extension 111. The thin walled metal tube 115 is flexible so that the introducer 100 can be advanced along a relatively tortuous vessel, such as a femoral artery, and so that the distal attachment region 102 can be longitudinally and rotationally manipulated. The thin walled metal tube 115 extends through the introducer 100 to the manipulation section 101, terminating at a connection means 116.

The connection means 116 is adapted to accept a syringe to facilitate the introduction of reagents into the thin walled metal tube 115. The thin walled metal tube 115 is in fluid communication with the apertures 112 of the flexible extension 111. Therefore, reagents introduced into connection means 116 will flow to and emanate from the apertures 112.

A plastic tube 141 is coaxial with and radially outside of the thin walled metal tube 115. The plastic tube 141 is “thick walled”—its wall is preferably several times thicker than that of the thin walled metal tube 1115. A sheath 130 is coaxial with and radially outside of the plastic tube 141. The thick walled plastic tube 141 and the sheath 130 extend distally to the manipulation region 101.

During the placement phase of the medical procedure, the prosthesis 120 is retained in a compressed condition by the sheath 130. The sheath 130 extends distally to a gripping and haemostatic sealing means 135 of the external manipulation section 101. During assembly of the introducer 100, the sheath 130 is advanced over the cylindrical sleeve 110 of the proximal attachment region 103 while the prosthesis 120 is held in a compressed state by an external force. A distal attachment (retention) section 140 (inside sheath 130 and not visible in this view) is coupled to the thick walled plastic tube 141. The distal attachment section 140 retains a distal end 142 of the prosthesis 120 during the procedure. Likewise, the cylindrical sleeve 110 retains the self-expanding zigzag stent 121.

The distal end 142 of the prosthesis 120 is retained by the distal attachment section 140. The distal end 142 of the prosthesis 120 has a loop (not shown) through which a distal trigger wire (not shown) extends. The distal trigger wire extends through an aperture (not shown) in the distal attachment section 140 into an annular region between the thin walled tube 115 and the thick walled tube 141. The distal trigger wire extends through the annular space to the manipulation region 101. The distal trigger wire exits the annular space at a distal wire release mechanism 125.

The external manipulation section 101 includes a haemostatic sealing means 135. The haemostatic sealing means 135 includes a haemostatic seal (not shown) and a side tube 129. The haemostatic sealing means 135 also includes a clamping collar (not shown) that clamps the sheath 130 to the haemostatic seal, and a silicone seal ring (not shown) that forms a haemostatic seal around the thick walled plastic tube 141. The side tube 129 facilitates the introduction of medical reagents between the thick walled tube 141 and the sheath 130.

A proximal portion of the external manipulation section 101 includes a release wire actuation section that has a body 136. The body 136 is mounted onto the thick walled plastic tube 141. The thin walled tube 115 passes through the body 136. The distal wire release mechanism 125 and the proximal wire release mechanism 124 are mounted for slidable movement onto the body 136.

The positioning of the proximal and distal wire release mechanisms 124 and 125 is such that the proximal wire release mechanism 124 must be moved before the distal wire release mechanism 125 can be moved. Therefore, the distal end 142 of the prosthesis 120 cannot be released until the self-expanding zigzag stent 121 has been released, and the barbs 151 have been anchored to the lumen. Clamping screws 137 prevent inadvertent early release of the prosthesis 120. A haemostatic seal (not shown) is included so that the release wires can extend out through the body 136 without unnecessary blood loss during the medical procedure.

A distal portion of the external manipulation section 101 includes a pin vise 139. The pin vise 139 is mounted onto the distal end of the body 136. The pin vise 139 has a screw cap 146. When screwed in, vise jaws (not shown) of the pin vise 139 clamp against or engage the thin walled metal tube 115. When the vise jaws are engaged, the thin walled tube 115 can only move with the body 136, and hence the thin walled tube 115 can only move with the thick walled tube 141. With the screw cap 146 tightened, the entire assembly can be moved together as one piece.

A second introducer may be used to introduce the tubular prosthesis 150. This second introducer may be based on the same principles as the introducer 100 described above, but less complex. For example, the second introducer may include a complex sheath for containing the tubular prosthesis 150 in a compressed state, so that it can be introduced into a targeted anatomy and then released to either self-expand or be actively expanded with a balloon.

The second introducer may also be adapted so that it can introduce the tubular prosthesis 150 by passing it through one ostium in the bifurcated prosthesis 120 and partially out of another ostium until the terminus of the tubular prosthesis 150 that is closest to the external end of the second introducer is properly positioned. At that point, the tubular prosthesis 150 can be released from the second introducer.

Prosthetic modules are preferably deployed seriatim. The intermodular connection between the tubular prosthesis 150 and the bifurcated prosthesis 120 is formed in situ. First the bifurcated prosthesis 120 is deployed, and then the tubular prosthesis 150 is deployed. For example, a bifurcated aortic prosthesis 120, as described in WO98/53761, can be deployed into the abdominal aorta. The bifurcated prosthesis 120 has a generally inverted Y-shaped configuration having a body portion 123, a shorter leg 160 and a longer leg 132. The body of the prosthesis is constructed from a tubular woven polyester. At the proximal end of the prosthesis 120 is a self-expanding stent 121 which extends beyond the end of the prosthesis and has distally extending barbs 151. The shorter leg 160 and the longer leg 132 have internal projections extending from their distal termini.

This bifurcated prosthesis 120 may be deployed in any method known in the art, preferably the method described in WO 98/53761, in which the device is inserted by an introducer via a surgical cut-down into a femoral artery, and then advanced into the desired position over a stiff wire guide using endoluminal interventional techniques. For example, a guide wire (not shown) is first introduced into a femoral artery of the patient and advanced until its tip is beyond the desired deployment region of the prosthesis 120. At this stage, the introducer assembly 100 is fully assembled, and ready for introduction into the patient. The prosthesis 120 is retained at one end by the cylindrical sleeve 110 and the other by the distal attachment sections 140, and compressed by the sheath 130. If an aortic aneurism is to be repaired, the introducer assembly 100 may be inserted through a femoral artery over the guide wire, and positioned by radiographic techniques, which are not discussed here.

Once the introducer assembly 100 is in the desired deployment position, the sheath 130 is withdrawn to just proximal of the distal attachment section 140. This action releases the middle portion of the prosthesis 120 so that it can expand radially. Consequently, the prosthesis 120 can still be rotated or lengthened or shortened for accurate positioning. The proximal self-expanding stent 121, however, is still retained within the cylindrical sleeve 110. Also, the distal end 142 of the prosthesis 120 is still retained within the external sheath 130.

Next, the pin vise 139 is released to allow small movements of the thin walled tube 115 with respect to the thick walled tube 141. These movements allow the prosthesis 120 to be lengthened or shortened or rotated or compressed for accurate placement in the desired location within the lumen. X-ray opaque markers (not shown) may be placed along the prosthesis 120 to assist with placement of the prosthesis.

When the proximal end of the prosthesis 120 is in place, the proximal trigger wire is withdrawn by distal movement of the proximal wire release mechanism 124. The proximal wire release mechanism 124 and the proximal trigger wire can be completely removed by passing the proximal wire release mechanism 124 over the pin vise 139, the screw cap 146, and the connection means 116.

Next, the screw cap 146 of the pin vise 139 is then loosened. After this loosening, the thin walled tube 115 can be pushed in a proximal direction to move the cylindrical sleeve 110 in a proximal direction. When the cylindrical sleeve 110 no longer surrounds the self-expanding stent 121, the self-expanding stent 121 expands. When the self-expanding stent 121 expands, the barbs 151 grip the walls of the lumen to hold the proximal end of the prosthesis 120 in place. From this stage on, the proximal end of the prosthesis 120 cannot be moved again.

Once the proximal end of the prosthesis 120 is anchored, the external sheath 130 is withdrawn distally of the distal attachment section 140. This withdrawal allows the contralateral limb 160 and the longer leg 132 of the prosthesis 120 to expand. At this point, the distal end 142 of the prosthesis 120 may still be moved. Such positioning of the prosthesis 120 may ensure that the shorter leg 160 extends in the direction of a contralateral artery.

After the shorter leg 160 extends in the direction of the contra-iliac artery, the tubular prosthesis 150 is deployed. The tubular prosthesis 150 is deployed such that it forms a connection with the shorter leg 160 and extends from the shorter leg 160 into the contralateral artery.

The embodiments described herein include external stents, but embodiments of the invention in which sutures that are impregnated or otherwise incorporate a reconstituted or naturally derived collagenous material are used to connect stent to a graft are not limited to external stents. Embodiments may include an internal or an anchoring stent, alone or with another stent.

Any other undisclosed or incidental details of the construction or composition of the various elements of the disclosed embodiment of the present invention are not believed to be critical to the achievement of the advantages of the present invention, so long as the elements possess the attributes needed for them to perform as disclosed. The selection of these and other details of construction are believed to be well within the ability of even one of rudimentary skills in this area, in view of the present disclosure.

Illustrative embodiments of the present invention have been described in considerable detail for the purpose of disclosing a practical, operative structure whereby the invention may be practiced advantageously. The designs described herein are intended to be exemplary only. The novel characteristics of the invention may be incorporated in other structural forms without departing from the spirit and scope of the invention. The invention encompasses embodiments both comprising and consisting of the elements described herein with reference to the illustrative embodiments. Unless otherwise indicates, all ordinary words and terms used herein shall take their customary meaning as defined in the New Shorter Oxford English Dictionary, 1993 edition. All technical terms not defined herein shall take on their customary meaning as established by the appropriate technical discipline utilized by those normally skilled in that particular art area. All medical terms shall take their meaning as defined by Stedman's Medical Dictionary, 27th edition.

It is therefore intended that the foregoing detailed description be regarded as illustrative rather than limiting, and that it be understood that it is the following claims, including all equivalents, that are intended to define the spirit and scope of this invention.

1. An implantable device comprising
a device component; and
at least one suture attached to the device component, the suture comprising a filament comprising a polymeric material and a naturally derived collagenous material.

2. An implantable device comprising
a device component; and
at least one suture attached to the device component, the suture comprising a plurality of filaments, wherein the filaments comprise a polymeric material and a naturally derived collagenous material.

3. The device of claim 2, wherein the suture is tied to the device component.

4. The device of claim 2, further comprising a second device component, the suture attaching the first and the second device components to each other.

5. The device of claim 2, wherein the device component comprises a stent.

6. The device of claim 2, wherein the device component comprises a graft.

7. The device of claim 2, wherein the naturally derived collagenous material is an extracellular matrix material selected from the group consisting of submucosa, renal capsule membrane, dermal collagen, dura mater, pericardium, fascia lata, serosa, peritoneum or basement membrane layers, liver basement membrane, intestinal submucosa, small intestinal submucosa, stomach submucosa, urinary bladder submucosa, and uterine submucosa.

8. The device of claim 2, wherein the filaments are coated with the naturally derived collageous material.

9. The device of claim 2, wherein the naturally derived collagenous material is made into at least one filament.

10. The device of claim 9, wherein the suture is a braided suture.

11. The device of claim 10, wherein some of the filaments comprise the polymeric material and other filaments comprise the naturally derived collagenous material.

12. The device of claim 11, wherein the filaments that comprise the polymeric material form a core of the braided suture.

13. The device of claim 12, wherein the filaments that comprise the naturally derived collagenous material are braided around the core of the braided suture.

14. The device of claim 12, wherein some other filaments that comprise the polymeric material and the filaments that comprise the naturally derived collagenous material are braided together around the core of the braided suture.

15. The device of claim 11, wherein some of the filaments that comprise the naturally derived collagenous material form a core of the braided suture.

16. The device of claim 15, wherein the filaments that comprise the polymeric material are braided around the core of the braided suture.

17. The device of claim 15, wherein some other filaments that comprise the naturally derived collagenous material and the filaments that comprise the polymeric material are braided together around the core of the braided suture.

18. The device of claim 2, wherein the suture is coated to improve its surface lubricity and knot tiedown behavior.

19. The device of claim 2, wherein the suture enhances adhesion of the device component to a tissue in place of contact of the suture with the tissue when used in a human body.

20. A suture comprising
(i) a filament comprising a polymeric material and a naturally derived collagenous material; or
(ii) a plurality of filaments, wherein the filaments comprise a polymeric material and naturally derived collagenous material.
